SEQUENCE LISTING (18 sequences)

SEQ ID NO: 1 - RNA, 52 nt, Artificial Sequence
  Synthetic Construct
ggauacggguaggaaaaacagcucauacccagcacauugaaaagcccuucuc

SEQ ID NO: 2 - RNA, 52 nt, Artificial Sequence
  Synthetic Construct
ggucgacauuagcaaaaccuacccguauccgagaagggcaaaagcacaucgu

SEQ ID NO: 3 - RNA, 52 nt, Artificial Sequence
  Synthetic Construct
ggcggacgaugacaaaagcuaaugccgaccacgaugugcaaaaacucgacua

SEQ ID NO: 4 - RNA, 52 nt, Artificial Sequence
  Synthetic Construct
ggcugccucaugcaaaagucaucguccgccuagucgaguaaaauccaacucc

SEQ ID NO: 5 - RNA, 52 nt, Artificial Sequence
  Synthetic Construct
gggugaacguugaaaaagcaugaggcagccggaguuggaaaaacugaugcac

SEQ ID NO: 6 - RNA, 52 nt, Artificial Sequence
  Synthetic Construct
ggguaugagcuguaaaaucaacguucacccgugcaucagaaaacaaugugcu

SEQ ID NO: 7 - RNA, 52 nt, Artificial Sequence
  Synthetic Construct
ggguaugagcuguaaaagucaucguccgccuagucgaguaaaacaaugugcu

SEQ ID NO: 8 - RNA, 52 nt, Artificial Sequence
  Synthetic Construct
ggguaugagcuguaaaagcaugaggcagccggaguuggaaaaacaaugugcu

SEQ ID NO: 9 - RNA, 52 nt, Artificial Sequence
  Synthetic Construct
ggucacaccuacaaaaaucaacguucacccgugcaucagaaaagugcuacac

SEQ ID NO: 10 - RNA, 52 nt, Artificial Sequence
  Synthetic Construct
ggguaugagcuguaaaauguaggugugaccguguagcacaaaacaaugugcu

SEQ ID NO: 11 - RNA, 52 nt, Artificial Sequence
  Synthetic Construct
ggaugcgugaggcaaaauguaggugugaccguguagcacaaaaguagagaug

SEQ ID NO: 12 - RNA, 52 nt, Artificial Sequence
  Synthetic Construct
ggguaugagcuguaaaagccucacgcaucccaucucuacaaaacaaugugcu

SEQ ID NO: 13 - RNA, 74 nt, Artificial Sequence
  Synthetic Construct
ggauacggguagguaugcgugaggaaaaauuaugcguggacagcucauacccagcacauugaaaagcccuucuc

SEQ ID NO: 14 - RNA, 74 nt, Artificial Sequence
  Synthetic Construct
ggucgacauuagcuagcgugacgaaaaaccucacgcauaccuacccguauccgagaagggcaaaagcacaucgu

SEQ ID NO: 15 - RNA, 74 nt, Artificial Sequence
  Synthetic Construct
ggcggacgaugacagcggucuuggaaaaucgucacgcuagcuaaugucgaccacgaugugcaaaaacucgacua

SEQ ID NO: 16 - RNA, 74 nt, Artificial Sequence
  Synthetic Construct
ggcugccucaugcaucuggagcuuaaaaccaagaccgcugucaucguccgccuagucgaguaaaauccaacucc

SEQ ID NO: 17 - RNA, 74 nt, Artificial Sequence
  Synthetic Construct
gggugaacguugacacucucauccaaaaaagcuccagaugcaugaggcagccggaguuggaaaaacugaugcac

SEQ ID NO: 18 - RNA, 74 nt, Artificial Sequence
  Synthetic Construct
ggguaugagcuguccacgcauaauaaaaggaugagagugucaacguucacccgugcaucagaaaacaaugugcu

(19) United States
(12) Patent Application Publication
(10) Pub. No.: US 2019/0093157 A1
(43) Pub. Date: Mar. 28, 2019

(54) RNA NANOTUBES FOR SINGLE MOLECULE SENSING AND DNA/RNA/PROTEIN SEQUENCING

(71) Applicant: Ohio State Innovation Foundation, Columbus, OH (US)

(72) Inventors: Peixuan Guo, Dublin, OH (US); Shaoying Wang, Middlesex, NJ (US); Hui Li, San Francisco, CA (US)

(21) Appl. No.: 16/091,741

(22) PCT Filed: Apr. 6, 2017
(86) PCT No.: PCT/US17/26418

§ 371 Date: Oct. 5, 2018

Related U.S. Application Data
(60) Provisional application No. 62/319,108, filed on Apr. 6, 2016.

(51) Int. Cl. C12Q 1/6869; C12Q 1/6806; C12Q 1/6837

(52) U.S. Cl. CPC C12Q 1/6869; B82Y 5/00; C12Q 1/6837; C12Q 1/6806

(57) ABSTRACT

Disclosed herein are RNA nanopores that can be used for single molecule sensing, disease diagnosis, and even protein sequencing.

### CROSS-REFERENCE TO RELATED APPLICATIONS

This application claims benefit of U.S. Provisional Application No. 62/319,108, filed Apr. 6, 2016, which is hereby incorporated herein by reference in its entirety.

### STATEMENT REGARDING FEDERALLY SPONSORED RESEARCH OR DEVELOPMENT

This invention was made with Government Support under grant numbers CA151648 and EB012135 awarded by the National Institutes of Health. The Government has certain rights to the invention.

### BACKGROUND

Nanopore technology has emerged as a cost-effective and high-throughput single molecule detection method, holding great potential for sensing and biopolymer sequencing over the last two decades. To date, many biological nanopore such as α-hemolysin, MspA, and viral connectors have been successfully used for incredibly versatile applications including detection of small molecules, macromolecule, polymers, polypeptides, as well as DNA and RNA. All of these nanopores come from biological system with defined channel size and physical property. Creating a new pore de novo with the ability to accurately tune the size and its functionality is still of great interest in fundamental and applied science. One of the key challenges in the de novo design of membrane channel is, to achieve a defined architecture in atomic scale with defined size, shape and properties.

By introducing polymerase or exonuclease, significant progress has been made in DNA sequencing using nanopore. However, so far there is still lack of effective way for protein sequencing which is critical for understanding its biological function in many physiology processes. One of possible method toward protein sequencing is to digest protein molecule into amino acids and discriminate them using nanopore sequentially. One of main obstacles is that short amino acids pass through the protein pore too fast and are hard to be detected with the nowadays available electronics.

### SUMMARY

Disclosed herein are RNA nanopores that can be used for single molecule sensing, disease diagnosis, and even protein sequencing. RNA strands are designed according to the disclosed methods such that they self-assemble to form a nanotube with hollow channel having a desired inside diameter and outsider diameter. The disclosed RNA nanopores have distinct advantages over natural and synthetic nanopores in the art. They have defined sizes, structure, and stoichiometry. Unpredictable side effects arising from heterogeneous nanopores can thus be avoided. RNA nanopores are easy to construct by self-assembly, they are highly soluble, and not prone to aggregation. The size and chemical properties of RNA nanopores can be easily tuned, e.g. using known RNA origami methods. RNA nanopores are thermodynamically stable and, therefore, the entire construct will remain intact at ultra-low concentrations. Finally, the polyvalent nature of RNA nanopores allows for easy integration of detection modules, targeting modules, imaging modules and therapeutic modules into a single form.

Also disclosed are methods of using the disclosed nanopores for single molecule sensing as well as DNA, RNA, and protein sequencing.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A and 1B illustrate the design of an origami designed RNA nanopore. FIG. 1A is a schematic top view (left) and side view (right) of an example 6-helix RNA nanopore structure. FIG. 1B is a map of an example RNA nanopore composed of 6 RNA strands. The two squares indicate two cholesterol molecules.

FIGS. 2A to 2C show the assembly and atomic force microscopy (AFM) of example origami designed RNA nanopores. FIG. 2A shows 4% Agarose gel electrophoresis of RNA nanopores with (FIG. 2A) and without (FIG. 2B) cholesterol. In FIG. 2A, Lane 1: Strand 1; Lane 2: Strands 1+2; Lane 3: Strands 1+2+3; Lane 4: Strands 1+2+3+4; Lane 5: Strands 1+2+3+4+5; Lane 6: Strands 1+2+3+4+5+6; Lane 7: DNA Ladder. In FIG. 2B, Lane 1: Strand 1; Lane 2: Strands 1+2; Lane 3: Strands 1+2+3; Lane 4: Strands 1+2+3+4; Lane 5: Strands 1+2+3+4+5; Lane 6: Strands 1+2+3+4+6; Lane 7: Strands 1+2+3+4+6+3′Chol-RNA; Lane 8: Strands 1+2+3+4+6+3′Chol-RNA+5′ Chol-RNA; Lane 9: DNA Ladder. FIG. 2C shows AFM analysis of RNA nanopore: representative individual RNA nanopore (left) and whole view of RNA nanopore (right).

FIGS. 3A and 3B show design and assembly of a six-helix RNA nanopore. FIG. 3A shows six nucleic acid sequence (SEQ ID NOs:1-6) when bound together by base paring form one face of a six-helix channel. FIG. 3B shows a 4% Agarose gel electrophoresis to confirm assemble of six bundled RNA nanopore. Lane 1: Strand 1. Lane 2: Strands 1+2. Lane 3: Strands 1+2+3. Lane 4: Strands 1+2+3+4. Lane 5: Strands 1+2+3+4+5. Lane 6: Strands 1+2+3+4+5+6. Lane 7: DNA Ladder. (b) 4% Agarose gel electrophoresis to confirm assemble of six bundled RNA nanopore with three cholesterol. Lane 1: Strand 1 with cholesterol (chol). Lane 2: Strands 1(Chol)+2. Lane 3: Strands 1(chol)+2+3(chol). Lane 4: Strands 1(chol)+2+3(chol)+4. Lane 5: Strands 1(chol)+2+3(chol)+4+5(chol). Lane 6: Strands 1(chol)+2+3(chol)+4+5(chol)+6. Lane 7: DNA Ladder.

FIG. 4 contains confocal images of LnCap cell after incubation with RNA nanopore containing cholesterol. RNA nanopores without cholesterol, single strand RNA and cell only used as the negative control as indicated in the image. The DAPI and Alexa 488 were used to stain the cell nucleuses and actin. Cy5 fluorophore conjugated these structure was used to track the interaction.

FIGS. 5A and 5B show characterization of origami designed RNA nanopore into lipid bilayer. FIG. 5A shows current trace of single channel insertion under 50 mV applied voltage. FIG. 5B shows conductance distribution of origami designed RNA nanopore. Condition: 1M KCl 5 mM Hepes, pH 8.

FIGS. 6A and 6B show characterization of six bundled RNA nanopore into lipid bilayer. FIG. 6A shows current trace of multiple RNA nanopore/liposome insertion under −50 mV applied voltage. FIG. 6B shows conductance distribution of RNA nanopore/liposome. Condition: 0.4 M KCl 5 mM Hepes, pH 8.

FIG. 7 shows typical current trace of poly arginine R2, R4, R6, R8, and R10 translocation through RNA nanopore. Condition:—50 mV, 0.4M KCl 5 mM Hepes, pH 8.

FIG. 8 shows dwell time distribution of poly arginine R2, R4, R6, R8, and R10 translocation through RNA nanopore. Condition:—50 mV, 0.4M KCl 5 mM Hepes, pH 8.

FIGS. 9A to 9E show sequences for example a 4-helix (FIG. 9A), a 5-helix (FIG. 9B), a 7-helix (FIG. 9C), an 8-helix (FIG. 9D), and a long 6-helix nanotube (FIG. 9E). FIG. 9A shows four nucleic acid sequence (SEQ ID NOs: 1-3, 7) that assemble into a 4-helix tube. FIG. 9B shows five nucleic acid sequence (SEQ ID NOs: 1-4, 8) that assemble into a 5-helix tube. FIG. 9C shows seven nucleic acid sequence (SEQ ID NOs: 1-5, 9-10) that assemble into a 7-helix tube. FIG. 9D shows eight nucleic acid sequence (SEQ ID NOs:1-5, 9, 11-12) that assemble into an 8-helix tube. FIG. 9E shows six nucleic acid sequence (SEQ ID NOs:13-18) that assemble into a long 6-helix tube.

FIGS. 10A and 10B are schematic top view (left) and side view (right) of a 4-helix (FIG. 10A) and an 8-helix (FIG. 10B) RNA nanopore structure.

FIG. 11 shows purification of RNA nanotube.

FIG. 12 shows magnified view of RNA nanotube viewed by atomic-force microscopy (AFM), with an estimated 11 nm×6 nm size.

### DETAILED DESCRIPTION

Nanopore-based analysis methods often involve passing a polymeric molecule, such as single-stranded DNA (“ssDNA”) or RNA, through a nanoscopic opening while monitoring a signal such as an electrical signal. Typically, the nanopore is designed to have a size that allows the polymer to pass only in a sequential, single file order. As the polymer molecule passes through the nanopore, differences in the chemical and physical properties of the monomeric units that make up the polymer, for example, the nucleotides that compose the ssDNA, are translated into characteristic electrical signals.

The signal can, for example, be detected as a modulation of the ionic current by the passage of an ssDNA molecule through the nanopore, which current is created by an applied voltage across the nanopore-bearing membrane or film. Because of structural differences between different nucleotides, different types of nucleotides interrupt the current in different ways, with each different type of nucleotide within the ssDNA producing a type-specific modulation in the current as it passes through a nanopore, and thus allowing the sequence of the DNA to be determined.

Nanopores that have been used for sequencing DNA include protein nanopores held within lipid bilayer membranes, such as α-hemolysin nanopores, and solid state nanopores formed, for example, by ion beam sculpting of a solid state thin film. Disclosed herein are RNA nanopores that provide advantages of the protein and solid state nanopores currently being used in these methods.

### Definitions

The term “RNA nanopore” and “RNA nanotube” are used herein interchangeably to refer to a nanostructure comprising one or more RNA strands that have self-assembled into a conformation having a hollow channel and at least one membrane-anchoring moiety.

The term “RNA” and “ribonucleic acid” refers to a natural or synthetic oligonucleotide or polynucleotide comprising two or more ribonucleotides linked by a phosphate group at the 3′ position of one ribonucleotide to the 5′ end of another ribonucleotide. Ribonucleic acids can also include nucleotide analogs (e.g., BrdU), and non-phosphodiester internucleoside linkages (e.g., peptide nucleic acid (PNA) or thiodiester linkages).

The term “RNA origami” refers to a method for organizing RNA molecules on the nanoscale, making it possible to fabricate complicated shapes from one or more strands of RNA. Unlike existing methods for folding DNA molecules, RNA origamis are produced by enzymes and simultaneously fold into pre-designed shapes. RNA molecules are strands that are composed of A, U, C and G nucleotides. A single strand of RNA can fold back on itself (or bind to another strand) by forming base pairs, interactions between individual nucleotides in the strand. The strongest base pairs in RNA are G-C, A-U and G-U, but many other base pairs can form in RNA as well.

Compositions

Nanopores

RNA strands are designed according to the disclosed methods such that they self-assemble to form a nanotube with hollow channel having a desired inside diameter and outsider diameter. RNA sequences for producing the disclosed RNA nanopores can be designed using known RNA origami techniques.

The design of RNA origamis can be done with assistance from computer algorithms. The designer can combine RNA helices and other 3D modules to form one interconnected strand using a 3D modeling environment. In this way the strand already has a set of sequence patterns defined, because the 3D modules constrain the sequence. Next, the strand is fed to a computer program that suggests the remaining A, U, C and Gs to assign to the rest of the structure, such that each part of the structure has a unique pattern that matches up. The program chooses the sequence from a very large space of solutions by testing many random sequences and then evaluating and comparing the energies of the base pairs from each input. After a target sequence is designed for a desired RNA, it can then be encoded into a DNA strand. When polymerase enzymes are added to the DNA genes, each copy of a DNA can be used to produce thousands of the encoded RNA structures.

In some embodiments, the nanotube comprises six RNA strands that assemble into six helices that form a hollow channel having an inside diameter of 1.7 nm and an outsider diameter of 6.3 nm. In some embodiments, the nanotube comprises four RNA strands that assemble into four helices that form a hollow channel having an inside diameter of 1 nm and an outsider diameter of 5.6 nm. In some embodiments, the nanotube comprises eight RNA strands that assemble into eight helices that form a hollow channel having an inside diameter of 3 nm and an outsider diameter of 7.6 nm.

In some embodiments, the nanopore comprises a plurality of RNA helices, wherein each helices is formed by complementary binding of at least two different RNA strands. As exemplified herein, this can be accomplished by designing RNA strands where each strand contains at least a first helix-forming region that is complementary with a helix-forming region of another RNA strand, and a second helix-forming region that is complementary with a helix-forming region of a different other RNA strand. It is important that each of these regions be complementary to only one RNA strand, i.e., no misannealing.

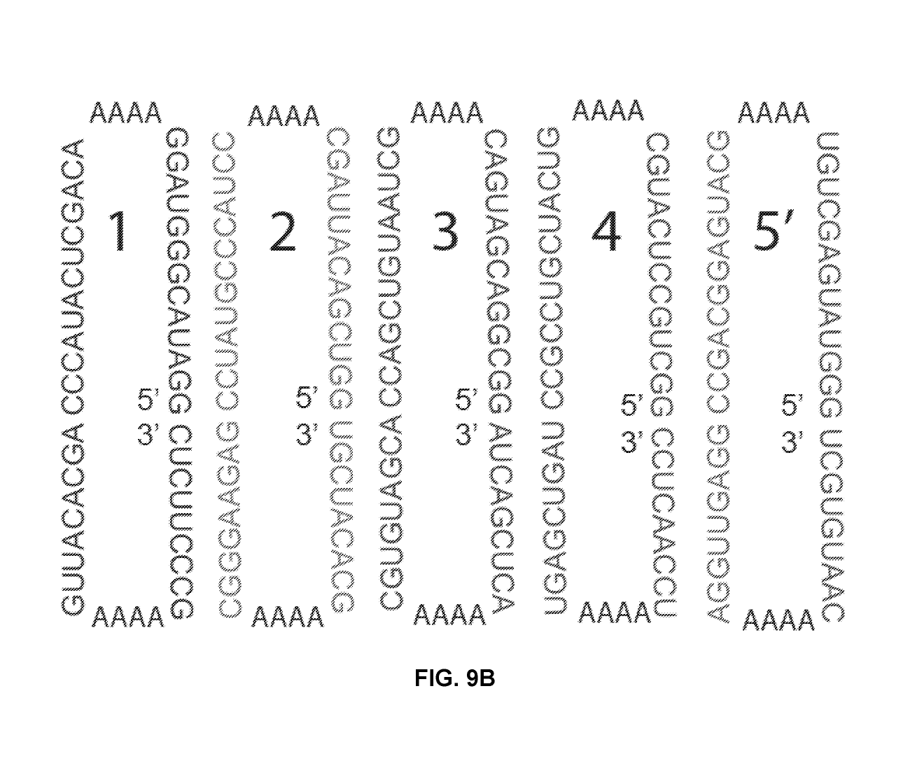

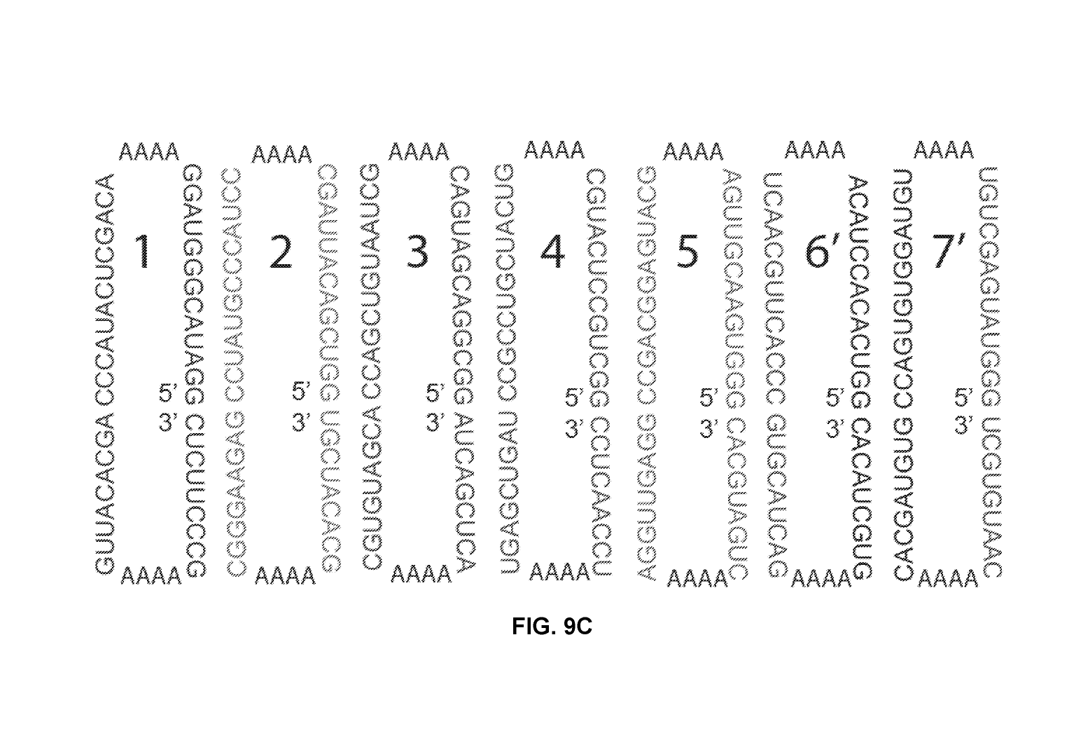

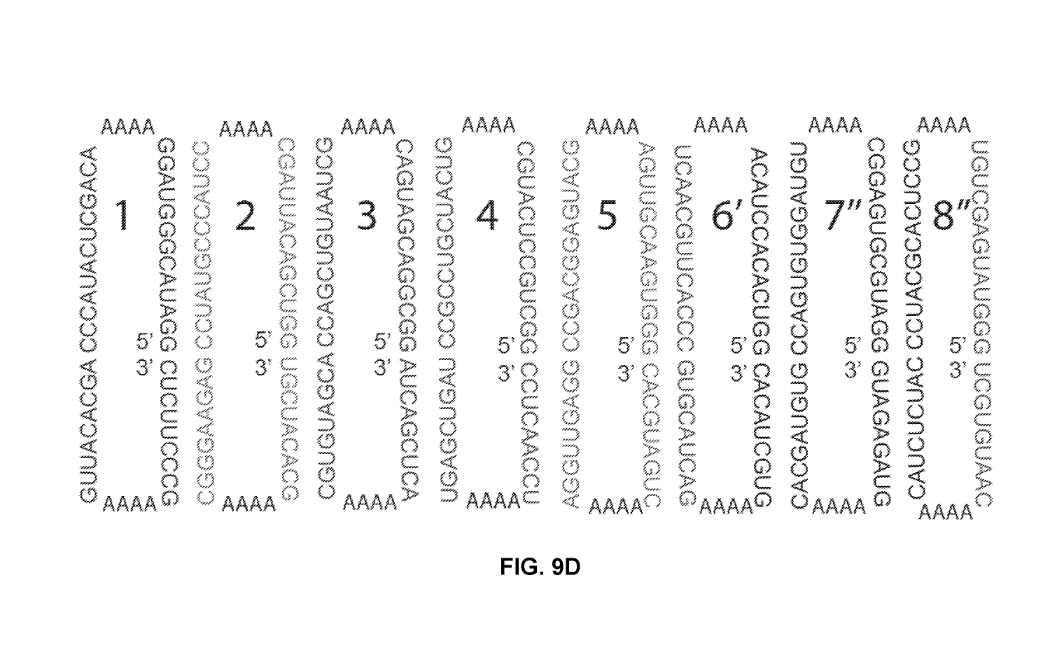

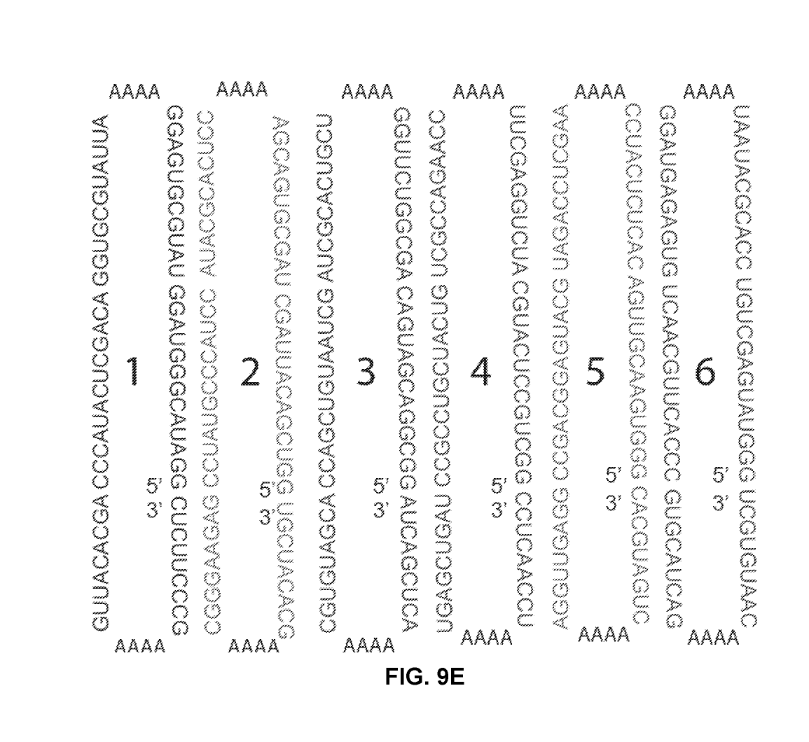

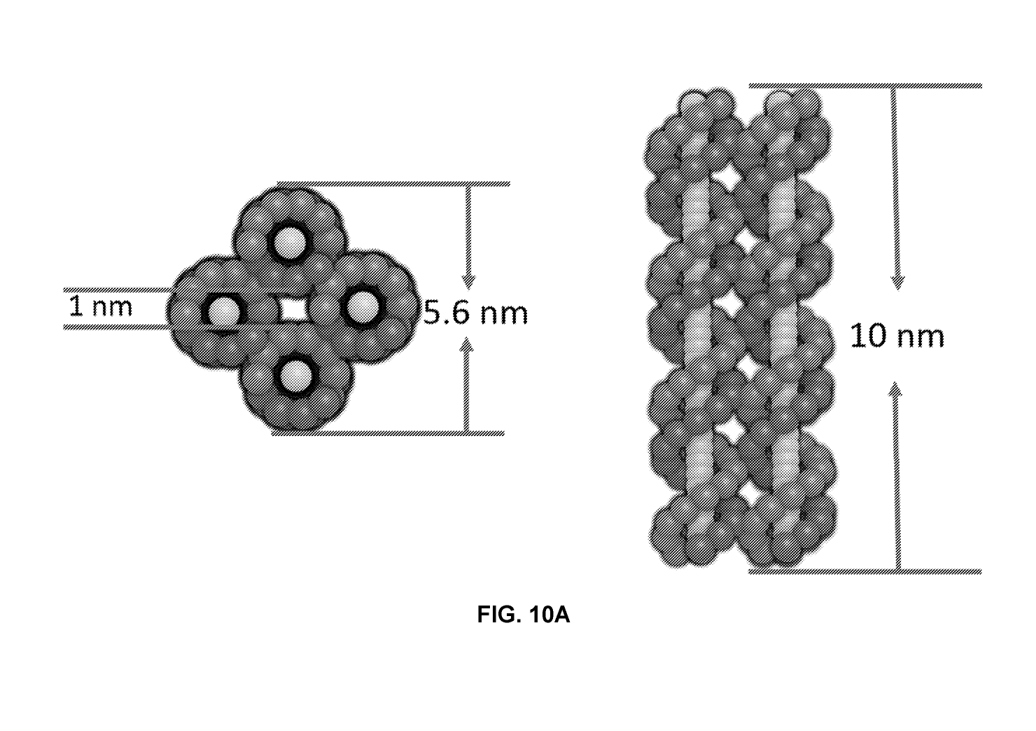

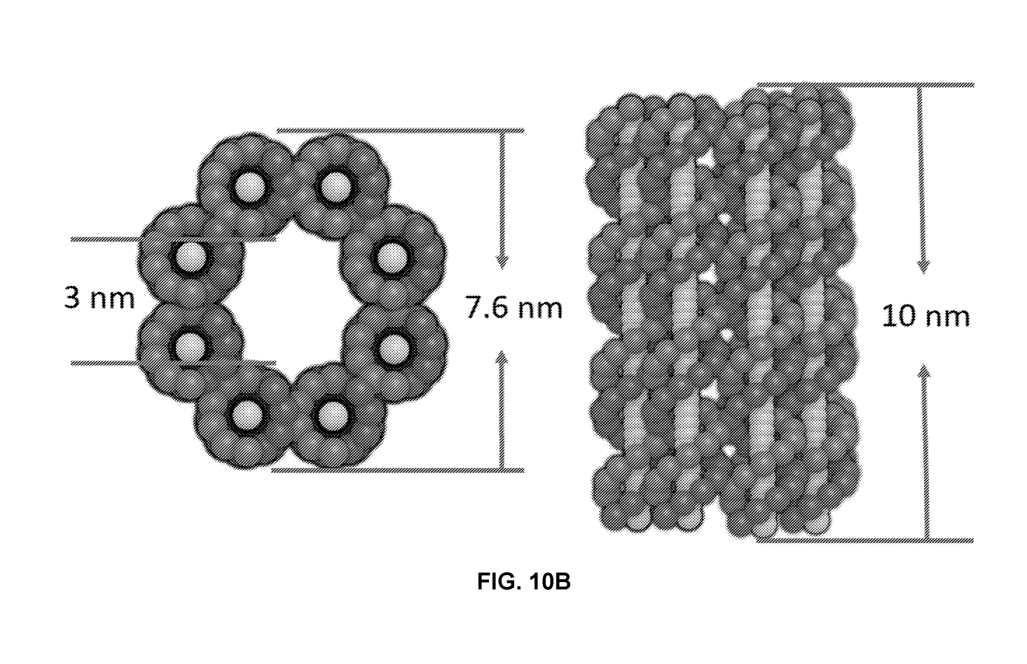

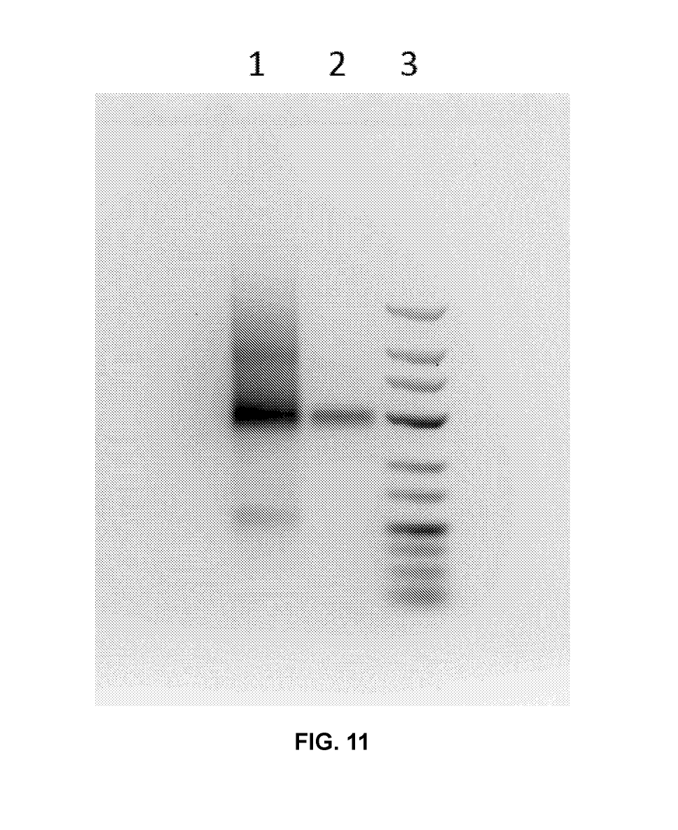

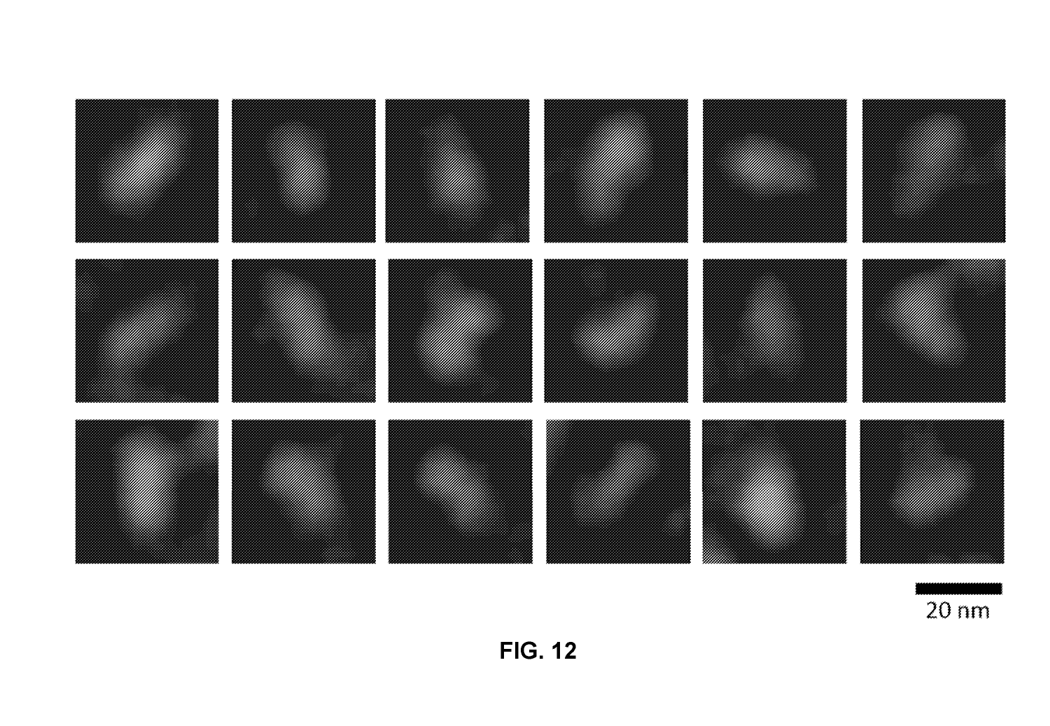

As an example, and as illustrated in FIG. 9A, a four-helix nanopore can be produced using four RNA strands, each having two helix-forming regions. The first strand can contain a first helix-forming region that is complementary to a second helix-forming region of the fourth strand and a second helix-forming region that is complementary to a first helix-forming region of the second strand. The second strand can contain a first helix-forming region that is complementary to the second helix-forming region of the first strand and a second helix-forming region that is complementary to a first helix-forming region of the third strand. The third strand can contain a first helix-forming region that is complementary to the second helix-forming region of the second strand and a second helix-forming region that is complementary to a first helix-forming region of the fourth strand. The fourth strand can contain a first helix-forming region that is complementary to the second helix-forming region of the third strand and a second helix-forming region that is complementary to a first helix-forming region of the first strand.

In some embodiments, the two helix-forming regions in an RNA strand are separated by a nucleic acid linker. In preferred embodiments, this linker is sized to provide a 180 degree bend. Therefore, in some embodiments, the linker comprises four nucleotides that are not complementary to any other sequence of any RNA strand. Non-limiting examples of suitable ribonucleotide linkers include AAAA, UUUU, UCUC, CUCU, UCCU, CUUC, AUUA, UAAU, CUUC, GUUG, CAAC, and GAAG. However, alternative linkers can be used according to known RNA origami method. In preferred embodiments, the linkers are selected to avoid complementarity with any RNA strand.

The nanopore can be designed to have any desired length. In some embodiments, the length of the nanopore is dependent on the thickness of the membrane it will transpose. In some embodiments, the nanopore is about 5 to 20 nm in length, including at about 5, 6, 7, 8, 9, 10, 15, or 20 nm in length.

The nanopore can be designed to have any suitable channel size. In some embodiments, channel diameter is selected to be just large enough for a single analyte to pass through. Therefore, the channel diameter can be dependent upon the size of the analyte. In some embodiments, the inner diameter of the nanopore is about 1 to 10 nm in diameter, including about 1, 2, 3, 4, 5, 6, 7, 8, 9, or 10 nm in diameter.

In some embodiments, at least one of the RNA strands comprises a membrane-anchoring moiety for incorporation into a lipid membrane. In some embodiments, the membrane-anchoring moiety comprises a cholesterol or modified cholesterol. Cholesterol is hydrophobic, and when conjugated to oligonucleotides, can facilitate uptake incorporation into cell membranes. In some embodiments, the cholesterol further comprises a triethylene glycol (TEG) spacer for conjugation to the oligonucleotide. Other lipophilic moieties capable of anchoring an oligonucleotide in the lipid bi-layer membrane can also be used. In some embodiments, cholesterol is added to the 5′ or 3′ end of 1, 2, 3, or more of the RNA strands.

In some embodiments, the disclosed nanopores further comprise one or more detection, targeting, imaging, or therapeutic moieties. For example, in some embodiments, the moieties are conjugated to one or more of the RNA strands. However, in some embodiments, the RNA strands used to produce the nanopores further comprise nucleic acid sequences that function as aptamers.

Methods

The disclosed RNA nanopores, when incorporated into a lipid membrane, may be useful for DNA and other polynucleotide sequencing. An electrolyte solution containing the DNA is placed on one side of the membrane. Electrolyte is also placed on the other side of the membrane. A voltage is applied through the electrolytes and across the membrane. This causes a DNA strand to gradually pass through the membrane. As the strand passes through, the current passing through the membrane is measured. The current is affected by the number and identity of the nucleotides presently in the pore. When using protein ion channels, there is typically more than one nucleotide in the pore. The identity of each nucleotide is determined from several current measurements as the nucleotide passes through the pore. In some embodiments, the disclosed nanopore has a length short enough to hold only one nucleotide. This can simplify the sequencing, as each nucleotide identification is determined from a single current measurement.

This approach can enable high selectivity, sensitivity, and real-time molecular recognition for a variety of target molecules of interest such as DNA, RNA, proteins, and ions. The proposed method can have the potential to sequence single stranded DNA (ssDNA) and RNA with resolution at the single base level, with similar capabilities for proteins (at the single amino acid level) and individual ions.

In some embodiments, a method is disclosed for sequencing a DNA, RNA, or protein sequence. This method can involve inserting an RNA nanotube into a lipid bilayer; inserting the lipid bilayer into an electrically resistive chip; applying an electric potential across the membrane to produce a current flowing only through the RNA nanotube; passing a single-stranded or double-stranded DNA, RNA, or protein strand through the RNA nanotube; and measuring a disruption in the current specific for each nucleotide or amino acid.

In some embodiments, a method is disclosed for detecting a molecule. This method can involve providing an RNA nanotube comprising a binding moiety specific for the molecule; inserting the RNA nanotube into a lipid bilayer; inserting the lipid bilayer into an electrically resistive chip; applying an electric potential across the membrane to produce a current flowing only through the RNA nanotube; and measuring a disruption in the current when the molecule is captured by the binding moiety of the RNA nanotube.

Nanopores hold promise for inexpensive, fast, and nearly “reagent-free” analysis of polymers. In a general embodiment of a nanopore system, an external voltage is applied across a nanometer-scale, electrolyte-filled pore, inducing an electric field. Any analyte, such as a polymer that contacts, resides in, or moves through, the interior of the pore, modulates the ionic current that passes through the pore depending on its physical characteristics. If the interior tunnel formed by the pore is of sufficiently small diameter and length, polymers that pass through must pass in a linear fashion, such that only a subset of the polymer subunits reside in the most constricted zone of the pore tunnel at one time. Thus, the ionic current fluctuates over time as the polymer passes through the nanopore, subunit by subunit, depending on the different physical characteristics of the subunit(s) residing in the nanopore constriction zone at each iterative step.

Therefore, the disclosed RNA nanopores can be “tuned” to have an inner diameter appropriate for the intended analyte. This tenability is a unique advantage of the disclosed nanopores.

Nanopores useful in the present disclosure include any pore capable of permitting the linear translocation of a polymer from one side to the other at a velocity amenable to monitoring techniques, such as techniques to detect current fluctuations.

In some cases, the RNA nanopore is disposed within a membrane, thin film, or lipid bilayer, which can separate a first and second conductive liquid media, which provides a nonconductive barrier between the first conductive liquid medium and the second conductive liquid medium. The nanopore thus provides liquid communication between the first and second conductive liquid media. In some embodiments, the nanopore provides the only liquid communication between the first and second conductive liquid media. The liquid media typically comprises electrolytes or ions that can flow from the first conductive liquid medium to the second conductive liquid medium through the interior of the nanopore. Liquids employable in methods described herein are well-known in the art. Descriptions and examples of such media, including conductive liquid media, are provided in U.S. Pat. No. 7,189,503, for example, which is incorporated herein by reference in its entirety. The first and second liquid media may be the same or different, and either one or both may comprise one or more of a salt, a detergent, or a buffer. Indeed, any liquid media described herein may comprise one or more of a salt, a detergent, or a buffer. Additionally, any liquid medium described herein may comprise a viscosity-altering substance or a velocity-altering substance.

In some embodiments, the analyte is capable of interacting with the nanopore. In the case of an analyte polymer, it can be capable of translocating, preferably in a linear fashion, through the nanopore to the other side. As used herein, the terms “interact” or “interacting,” indicate that the analyte moves at least to the opening of the nanopore and, optionally, moves through the nanopore. As used herein, the terms “through the nanopore” or “translocate” are used to convey for at least some portion of the polymer analyte to enter one side of the nanopore and move to and out of the other side of the nanopore. In some cases, the first and second conductive liquid media located on either side of the nanopore are referred to as being on the cis and trans regions, where the analyte polymer to be measured generally translocates from the cis region to the trans region through the nanopore. However, in some embodiments, the analyte polymer to be measured can translocate from the trans region to the cis region through the nanopore. In some cases, the entire length of the polymer does not pass through the pore, but portions or segments of the polymer pass through the nanopore for analysis.

The analyte polymer can be translocated through the nanopore using a variety of mechanisms. For example, the analyte polymer and/or reference sequence can be electrophoretically translocated through the nanopore. Nanopore systems also incorporate structural elements to apply an electrical field across the nanopore-bearing membrane or film. For example, the system can include a pair of drive electrodes that drive current through the nanopores. Additionally, the system can include one or more measurement electrodes that measure the current through the nanopore. These can be, for example, a patch-clamp amplifier or a data acquisition device. For example, nanopore systems can include an Axopatch-1B patch-clamp amplifier (Axon Instruments, Union City, Calif.) to apply voltage across the bilayer and measure the ionic current flowing through the nanopore. The electrical field is sufficient to translocate a polymer analyte through the nanopore. As will be understood, the voltage range that can be used can depend on the type of nanopore system being used. For example, in some embodiments, the applied electrical field is between about 20 mV and about 260 mV, for protein-based nanopores embedded in lipid membranes. In some embodiments, the applied electrical field is between about 40 mV and about 200 mV. In some embodiments, the applied electrical field is between about 100 mV and about 200 mV. In some embodiments, the applied electrical field is about 180 mV. In other embodiments where solid state nanopores are used, the applied electrical field can be in a similar range as described, up to as high as 1 V.

Additionally or alternatively, nanopore systems can include a component that translocates a polymer through the nanopore enzymatically. For example, a molecular motor can be included to influence the translocation of polymers through the nanopore. A molecular motor can be useful for facilitating entry of a polymer into the nanopore and/or facilitating or modulating translocation of the polymer through the nanopore. Ideally, the translocation velocity, or an average translocation velocity, is less than the translocation velocity that would occur without the molecular motor. In any embodiment herein, the molecular motor can be an enzyme, such as a polymerase, an exonuclease, or a Klenow fragment. In one example, described in more detail below, a DNA polymerase such as phi29 can be used to facilitate movement in both directions.

Characteristics of an analyte, or subunits thereof, can be determined in a nanopore system based on measurable effects of their residency in the nanopore. In some embodiments, the mere presence of the analyte, e.g. the presence of one or more polymer subunits, is confirmed in the analysis. In some embodiments, additional information can be determined about the one or more polymer subunits in the analyte domain. In some embodiments, the presence of an identifiable characteristic, such as a “fingerprint” or primary subunit sequence, is identified in the analyte domain. In some embodiments, the sequence identity is determined for one, two, or more polymer subunits in the analyte domain. In some embodiments, the sequence of the analyte domain is determined.

Characteristics of the analyte domain, or subunit(s) thereof, can be determined based on the effect of the analyte domain, or subunit(s) thereof, on a measurable signal when interacting with the nanopore. The output signal produced by the nanopore system is any measurable signal that provides a multitude of distinct and reproducible signals depending on the physical characteristics of the polymer or polymer subunit(s). For example, the ionic current level through the pore is an output signal that can vary depending on the particular polymer subunit(s) residing in the constriction zone of the nanopore. As the polymer translocates in iterative steps (e.g., linearly, subunit by subunit through the pore), the current levels can vary to create a trace, or “current pattern,” of multiple output signals corresponding to the contiguous sequence of the polymer subunits. This detection of current levels, or “blockade” events have been used to characterize a host of information about the structure polymers, such as DNA, passing through, or held in, a nanopore in various contexts.

In another aspect, the disclosure provides a method of sequencing two or more nucleotides of a nucleic acid, comprising: (a) providing a nucleic acid comprising at least two unknown nucleotides in an analyte domain, the nucleic acid further comprising a positively or negatively charged moiety in an end domain at the 3′ or 5′ end; (b) providing an RNA nanopore positioned between a cis side, comprising a first conductive liquid medium and the modified nucleic acid, and a trans side, comprising a second conductive liquid medium; and (c) causing the nucleic acid to pass through a tunnel of the nanopore, thereby producing a first and a second ion current level, thereby sequencing two or more nucleotides of the nucleic acid.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Biomimetic RNA-Based Nanopore for Short Peptide Detection

Materials and Methods

Materials

The phospholipid 1, 2-diphytanoyl-sn-glycerol-3-phosphocholine (DPhPC) was obtained from Avanti Polar Lipids, Inc. Organic solvents (n-decane and chloroform) were purchased from Fisher Scientific, Inc. and TEDIA, Inc., respectively. Poly arginine with 2, 4, 6, 8, 10 amino acids peptide was custom-ordered from GenScript, Inc. n-octyl-oligo-oxyethylene was purchased from Enzo company. All other reagents were purchased from Sigma or Fisher, if not specified.

Design, Synthesis, and Self-Assembly of RNA Nanopores

The NanoEngineer program was used to facilitate the design of the RNA tubes. Unmodified RNA strands were synthesized by in vitro T7 transcription and purified by 8 M urea, 8% denaturing PAGE. DNA templates for transcribing the RNA strands were made by polymerase chain reactions (PCR). DNA oligonucleotides for PCR were directly ordered from IDT, Inc. Cholesterol modified RNA strands were ordered from IDT or synthesized by Azco Oligo-800 DNA and RNA synthesizer.

The RNA tubes were self-assembled in a one-pot manner by mixing the synthesized RNA strands in equimolar concentrations in 1×TMS buffer and heated to 95° C. and slowly cool down to room temperature. The step-wise self-assembly of the RNA tubes was examined by agarose gels. The gels were stained with Ethidium Bromide (EB) and imaged by Typhoon FLA 7000 (GE Healthcare).

Atomic Force Microscopy

The RNA tubes were imaged by atomic force microscopy (AFM). RNA tubes were placed on the APS-modified mica surface and excess samples were washed with DEPC water and dried before imaging. AFM imaging was performed by using the MultiMode AFM NanoScope IV system (Veeco) operated in tapping mode.

In Vitro Binding of RNA Nanopores to Cancer Cells

Prostate cancer LnCap cells were grown on glass cover slides in 24-well plates in RPMI-1640 medium with 10% FBS at 37° C. in humidified air containing 5% CO₂ overnight. RNA nanopores harboring chosterol and labeled by Cy5 were diluted in optium-MEM medium to 100 nM and incubated with the cells for 2 hrs at 37° C. RNA nanopores without cholesterol and single strand RNA were used as the negative control. After the incubation, the cells were washed with PBS and fixed by 4% paraformaldehyde. Alexa488 Phalloidin (Life Technologies) was used to stain the cellular actin. The Prolong Gold antifade reagent with DAPI (Life Technologies) were then used to stain the cell nucleuses and mount the cells to the glass slides. The confocal images were recorded by the FluoView FV1000-Filter Confocal Microscope System (Olympus).

Insertion of the RNA Nanopore into Planar Lipid Bilayer

Briefly, planar bilayer lipid membranes (BLMs) were generated in a BCH-1A horizontal BLM cell (Eastern Scientific). A Teflon partition with a 200 μm aperture was placed in the apparatus to separate the BLM cell into cis- (top) and trans- (bottom) compartments. A planar lipid bilayer was formed by painting the aperture with 0.5 μL of 3% (w/v) DPhPC in n-decane. A conducting buffer (1 M KCl, 5 mM HEPES, pH 7.8) was added to both the top and bottom compartments of the BLM cell, and Ag/AgCl electrodes were placed in the buffer of each compartment. The electrode in the trans-compartment was connected to the headstage of an Axopatch 200B amplifier (Axon Instruments, Inc.), and the electrode in the top compartment was grounded.

For direct insertion, a mixture of cholesterol-anchored RNA nanopores and 0.5% n-octyl-oligo-oxyethylene dissolved in conducting buffer was added to the cis side of the bilayer to a final concentration of 100 nM nanopores. For pore insertion with liposome, a two-step procedure was employed for this design. DPhPC lipids in chloroform were dehydrated to eliminate solvents first and then rehydrated with buffer containing 250 mM sucrose and purified RNA nanopore with a final concentration 250 nM. The multilamellar lipid-RNA nanopore suspension was then extruded through 400 nm polycarbonate membrane filters to generate uniform unilamellar liposomes with the RNA nanopore embedded in the membrane. The resulting liposome-RNA nanopore complex was fused with a planar lipid membrane to generate planar membrane-embedded RNA nanopore.

Electrophysiological Measurements

The headstage and Axopatch 200B patch clamp amplifier were connected to a DigiData 1440 analog-digital converter (Axon Instruments, Inc.) to monitor and record electrochemical currents through BLMs. The current recordings were low-pass filtered at a frequency of 5 kHz. The sampling frequency was 20 kHz in all experiments, unless otherwise specified. The data were recorded a with pClamp 9.1 software (Axon Instruments, Inc.). The blockades were calculated with a home-built Matlab program and statistically analyzed with OriginPro 8.1 (OriginLab Corporation). The capture rate was derived by exponentially fitting the time interval between two adjacent events as described previously. For peptide translocation experiments, poly arginine was premixed with conducting buffer with a final concentration of 1 μg/mL before the insertion of RNA nanopore.

Results

Design, Synthesis, and Self-Assembly of RNA Nanopores

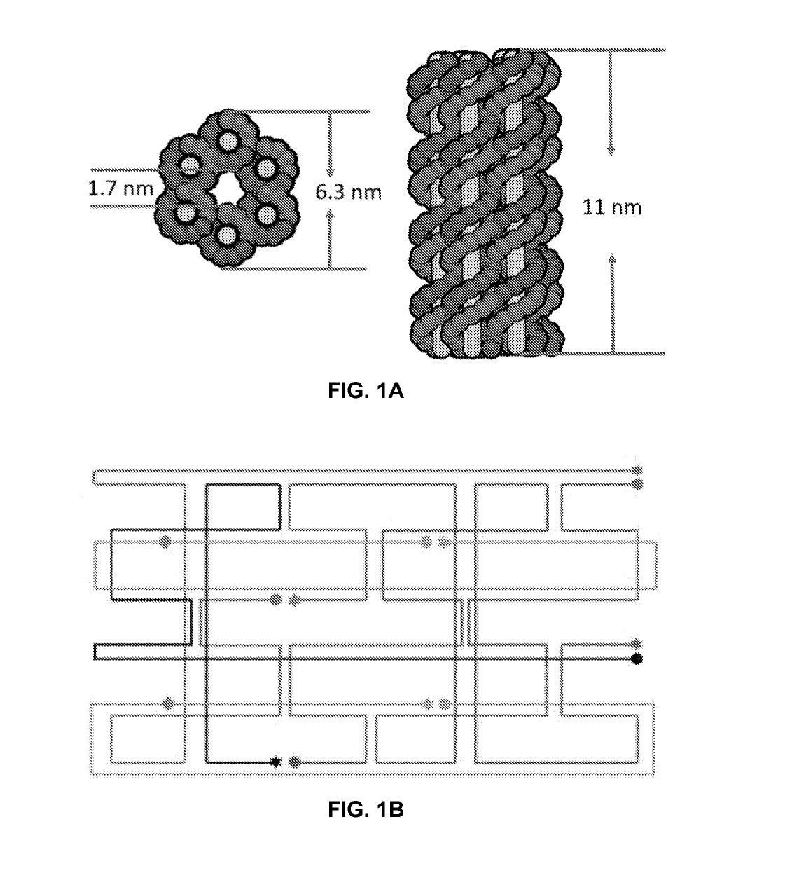

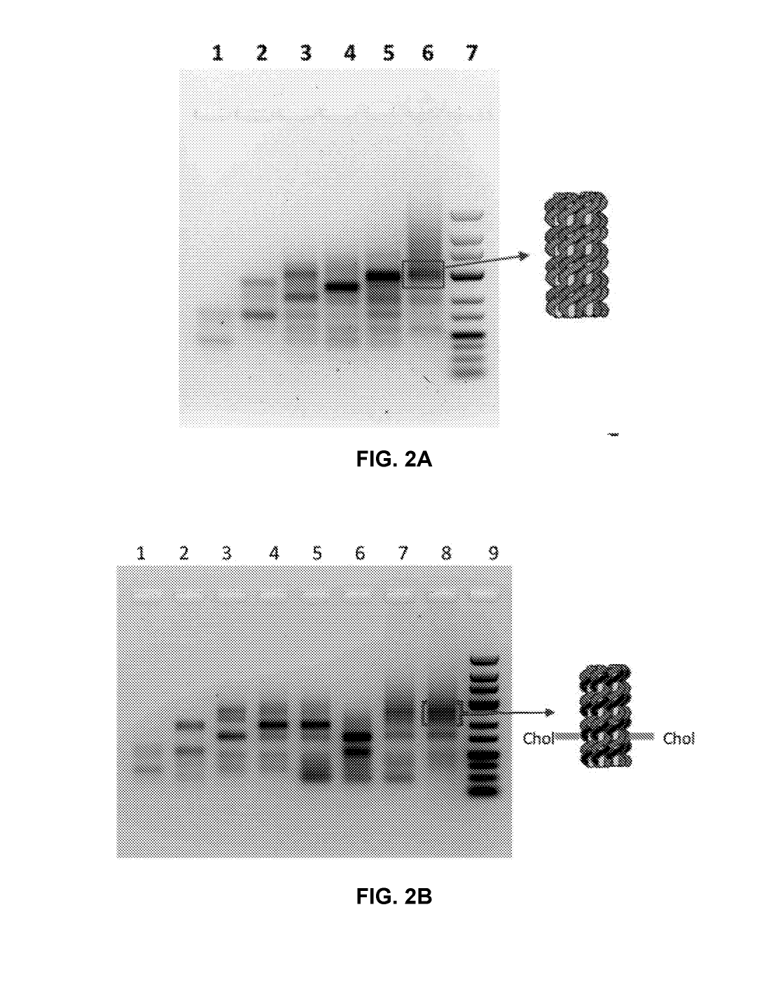

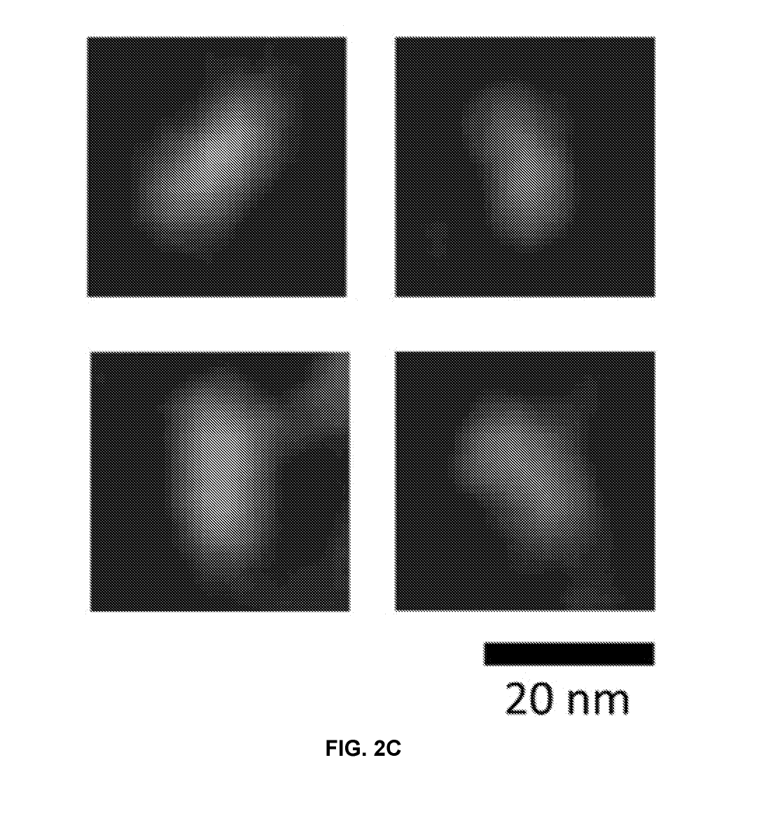

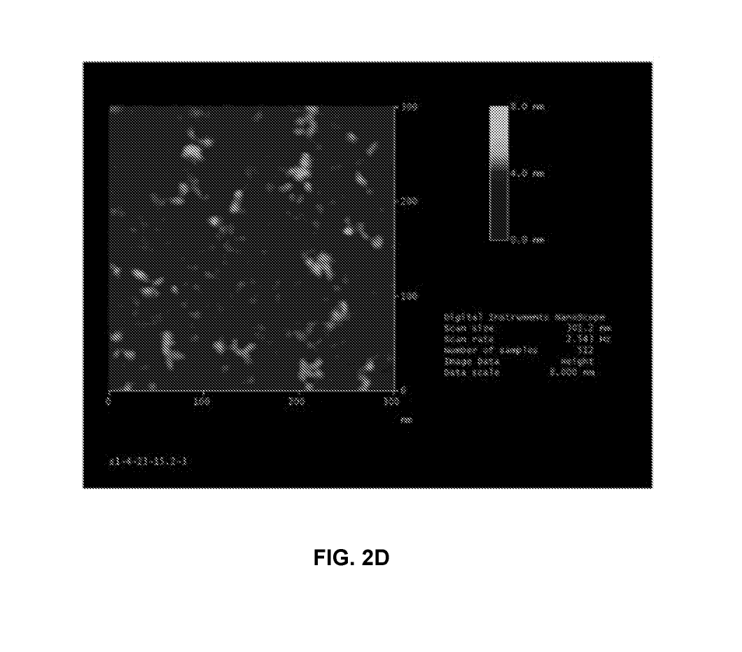

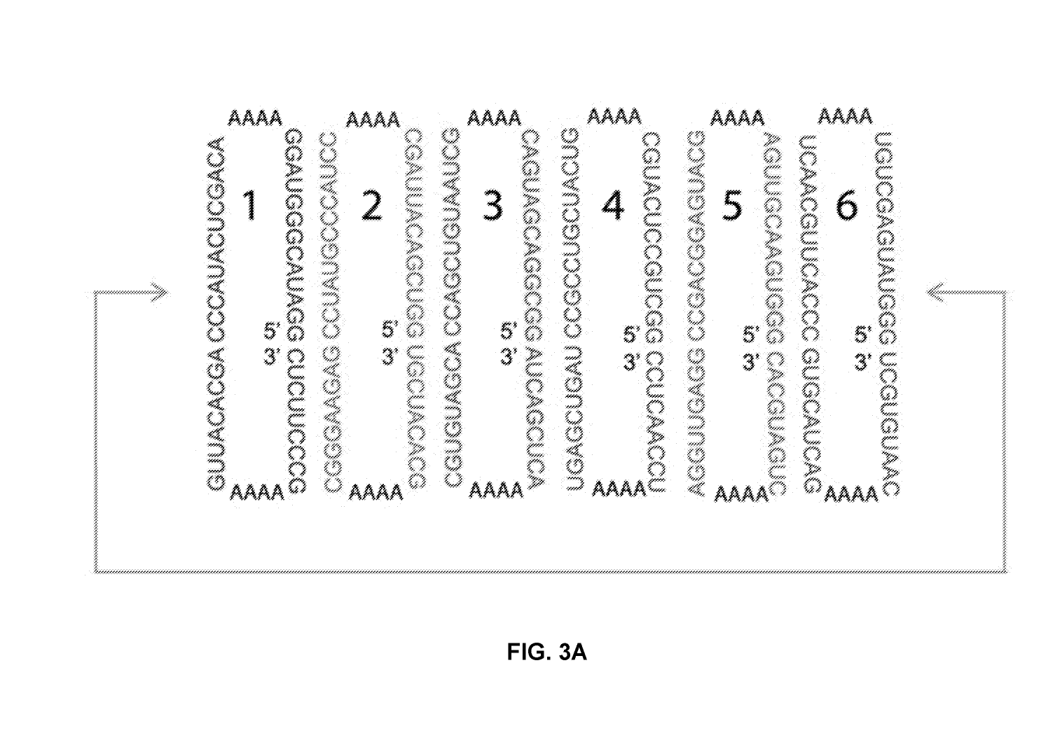

RNA tubes were designed using NanoEngineer software. In the first RNA nanopore design, six RNA strands were used to assemble the tube by utilizing a RNA origami approach similar to DNA origami (FIG. 1). Six RNA strands were designed to fold together to form a hollow channel with the insider diameter of 1.7 nm and the outsider diameter of 6.3 nm. To assemble the RNA nanopore with cholesterol, two more chemically synthesized RNA strands with cholesterol at the end were added to the structure. For assembling, the synthesized RNA strands were mixed in stoichiometric ratio and annealed in 1×TMS buffer in a one-pot manner. Step-wise self-assembly of the RNA nanopores with and without cholesterol was examined by agarose gels, confirming the formation of the high molecular weight RNA nanopores (FIG. 2).

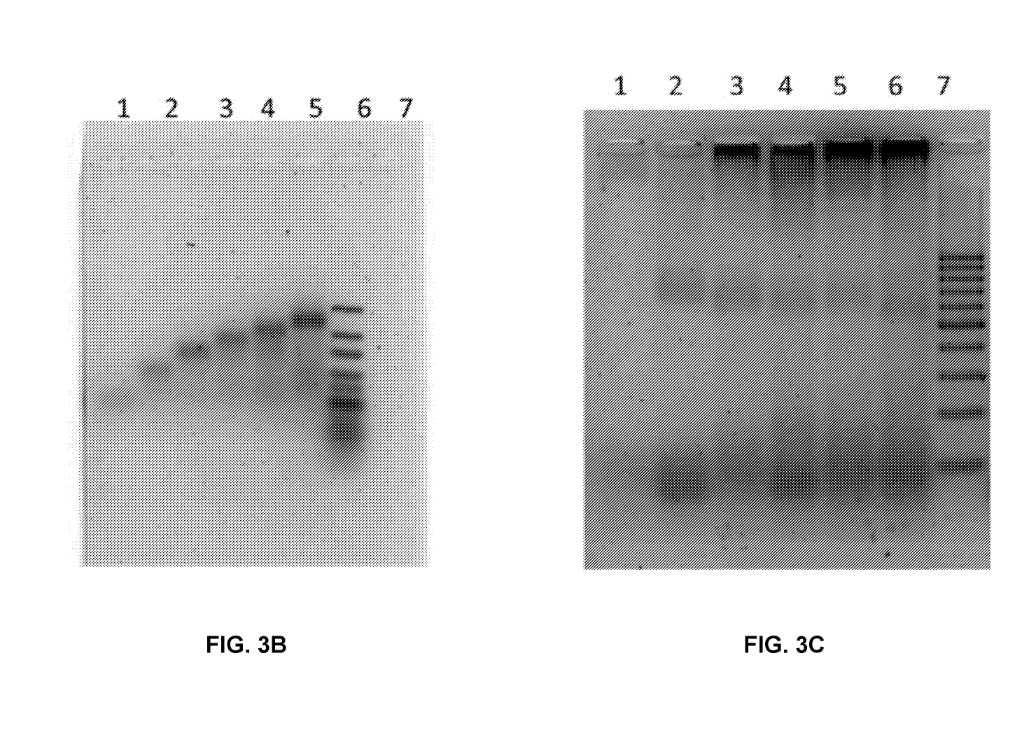

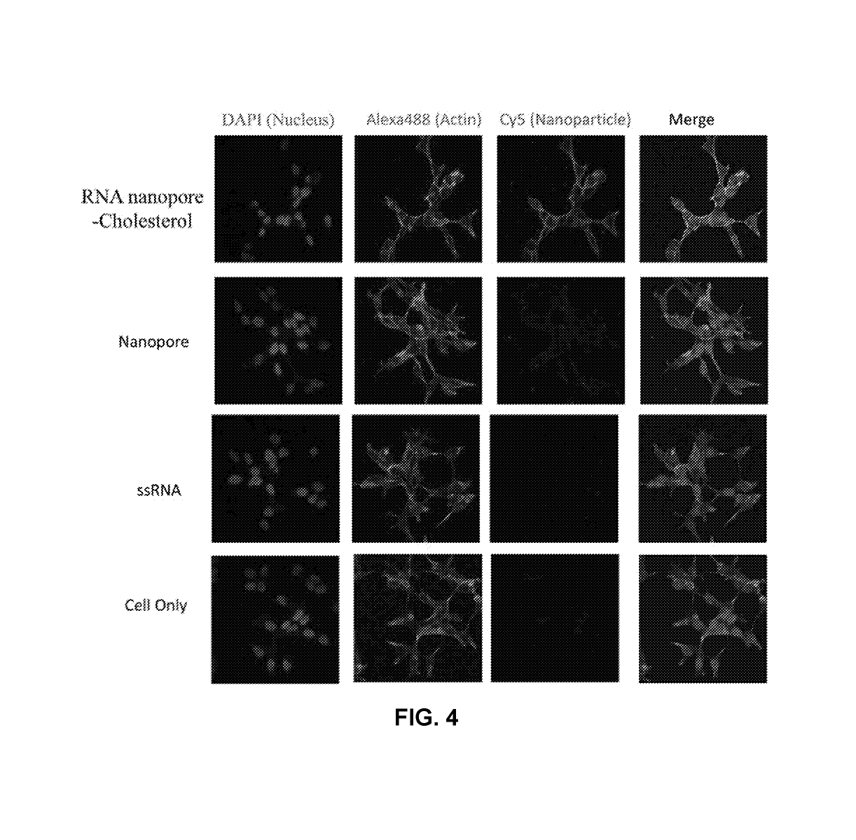

For the second RNA nanopore design with three cholesterol molecules, six RNA strands were designed to be bound together by base paring and each RNA strand form one face of the six-helix channel. Similar channel size with the first RNA nanopore design was predicted, since both designs were composed of six RNA helixes bound together (FIG. 3A). Compared with previous design, this approach has several advantages, such as ease of fabrication, higher yield of pores at higher concentration, and lower synthesis cost. FIG. 3B shows RNA nanopore without cholesterol can successfully assemble high molecular weight RNA nanopores step-wisely. However, after incorporate the three strands with cholesterol molecules, the assembled structure cannot run into gel and stuck in the loading well. This is possibly due to the formation of aggregated structure resulted from the hydrophobic interaction between cholesterol molecules (FIG. 4C).

Atomic Force Microscope Characterization of RNA Nanopores

Atomic force microscope was used to characterize the RNA nanopores. As shown in the AFM images, individual RNA nanopore can be clearly observed under AFM (FIGS. 2C, 2D). The observed RNA nanopores under atomic force microscope size and shape is in agreement to the design, confirming the successful self-assembly of RNA nanopores.

Characterization of RNA Nanopore Insertion into Cellular and Lipid Bilayer Membrane

To study whether RNA nanopore can interact with cellular membrane, RNA nanopore with and without anchored cholesterol and several other controls, were incubated with LnCap cell. Cy5 fluorophore was conjugated to these structures to track the interaction. The results have shown only cholesterol anchored RNA nanopore group displayed strong cy5 signal in cell membrane indicating cholesterol anchored RNA nanopore group inserted into the cell membrane (FIG. 4). Similar results has also been verified in giant unilamellar vesicles that only RNA nanopore with anchored cholesterol can insert into the lipid membrane of giant unilamellar vesicles.

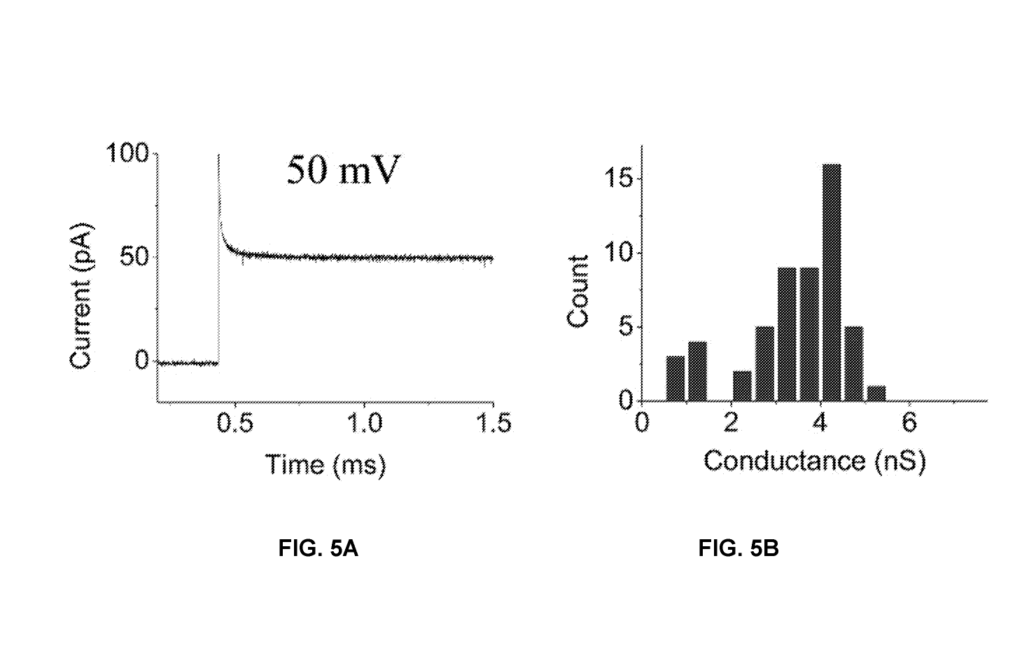

To incorporate origami designed RNA nanopore into planar lipid membranes, direct insertion of cholesterol-anchored RNA nanopore with detergent n-octyl-oligo-oxyethylene was first tried but no insertion was observed. This may be because the hydrophobicity of two cholesterol was not strong enough to overcome the energy barrier of inserting into lipid bilayer. Therefore, a two-step procedure was employed for this design. Briefly, DPhPC lipids in chloroform were dehydrated to eliminate solvents first and then rehydrated with buffer containing 250 mM sucrose and purified origami designed RNA nanopore with a final concentration 250 nM. The multilamellar lipid-RNA nanopore suspension was then extruded through 400 nm polycarbonate membrane filters to generate uniform unilamellar liposomes with the RNA nanopore embedded in the membrane. The resulting liposome-RNA nanopore complex was fused with a planar lipid membrane to generate planar membrane-embedded RNA nanopore. Electrophysiological properties of membrane-embedded RNA nanopore was characterized by single channel conductance assay. As revealed in a continuous current trace, the RNA nanopore insertion were observed (FIG. 5A). The insertion of single RNA nanopore results in various current jump under an applied potential of 50 mV in conducting buffer (1 M KCl, 5 mM HEPES, pH 8). As revealed in FIG. 5B, the conductance distribution of is wide. This wide distribution is attributed to simultaneous insertion of multiple RNA nanopore or multiple RNA nanopore aggregated together.

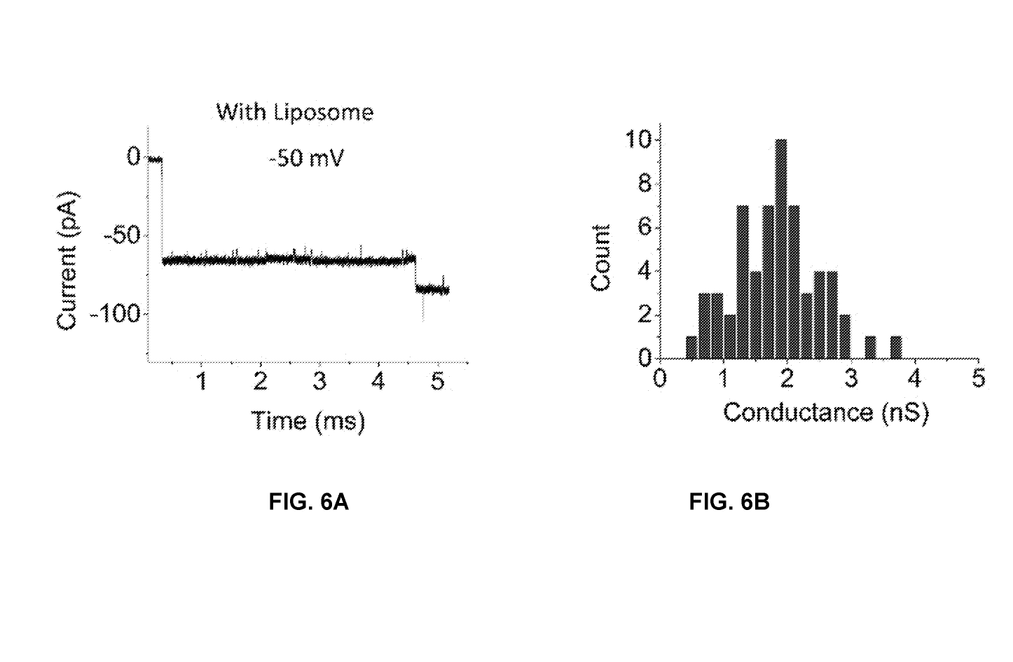

Due to the insertion rate of RNA nanopore is relative low with two anchored cholesterol, another design with three anchored cholesterol and a bundle of six RNA duplexes was constructed. Direct incubation of the three anchored cholesterol RNA nanopore with planar lipid bilayer has resulted distinguish stepwise current jump (FIG. 6B). Two step prepared liposome-RNA nanopore complex has also resulted in similar stepwise current jump (FIG. 6A). The conductance distribution of this RNA nanopore is also wide under 0.4M KCl, 5 mM Hepes, pH8 (FIGS. 6C, 6D). The wide distribution of this RNA nanopore is also possibly due to multiple nanopores aggregated together cholesterol group. This is also revealed in the assemble gel in which the anchored cholesterol RNA nanopore stuck in the well.

Characterization of Peptide Translocation Through RNA Nanopore

One possible way toward protein sequencing is to digest protein into amino acids and discriminate them sequentially using nanopore. However, detection of short amino acids by nanopore is still challenging due to the ultra-fast translocation speed. In this study, translocation of poly arginine with 2, 4, 6, 8, 10 amino acids through RNA nanopore were measured and compared.

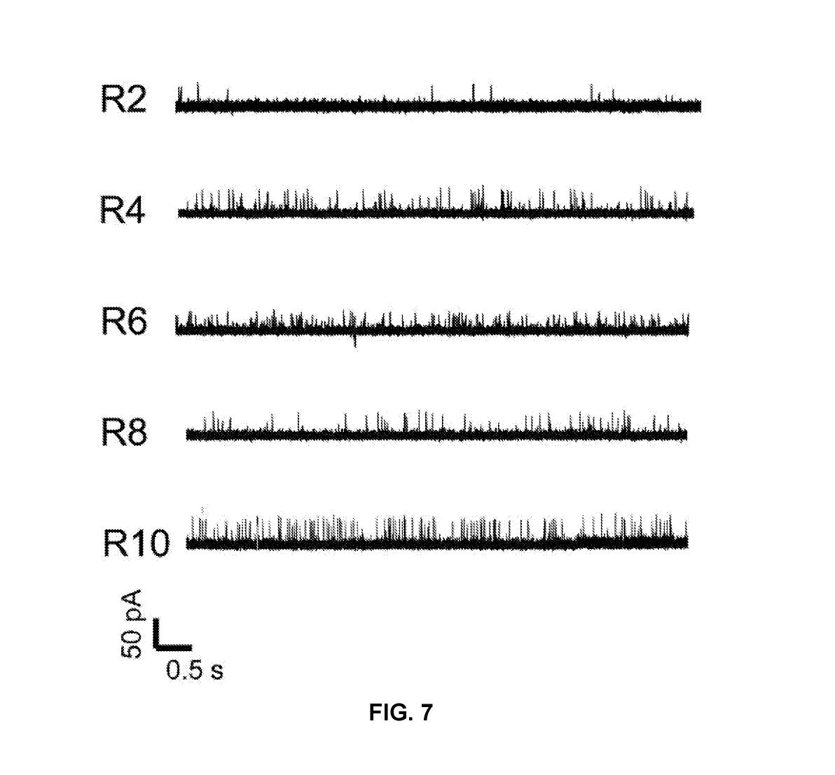

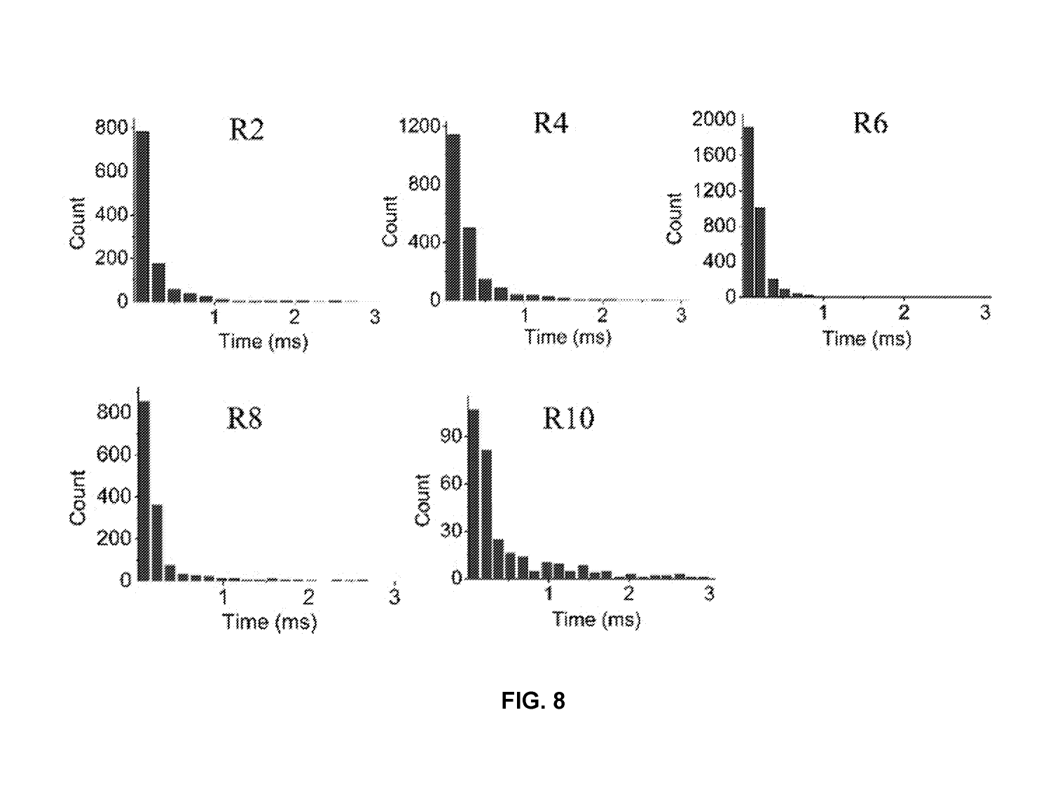

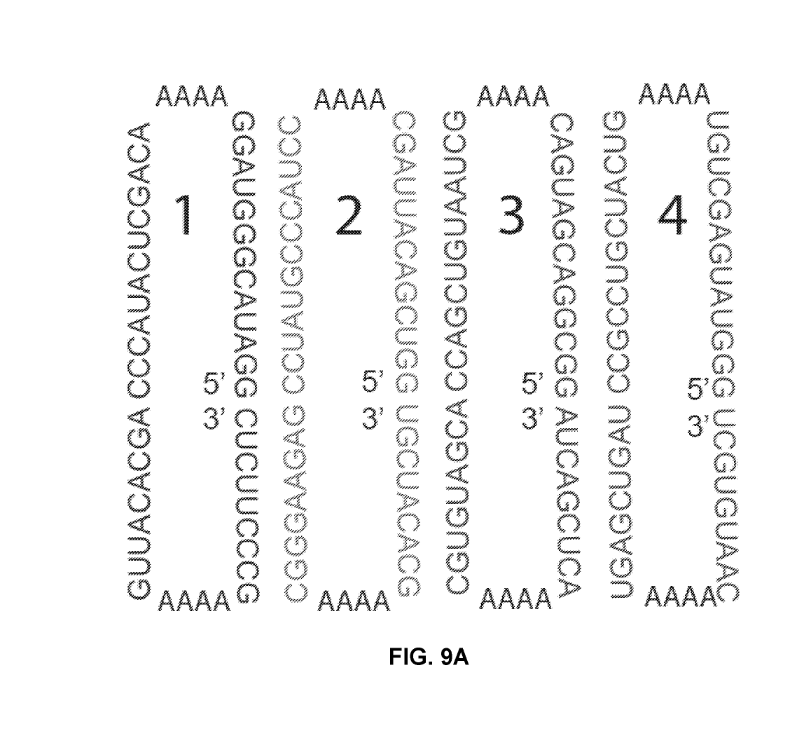

FIG. 7 presents typical current trace of R2, R4, R6, R8, and R10 translocation through RNA nanopore with negative 50 mV applied voltage. All of these peptides has displayed similar current blockade. As shown in FIG. 7, R2 has displayed lower capture rate compared with other peptides, which may be because the translocation speed is too fast for R2 and many events has been filtered. The dwell time for each peptides was derived. The dwell time of R2, R4, R6, R8, and R10 were 0.14±0.01, 0.22±0.01, 0.17±0.01, 0.15±0.01, and 0.28±0.03 ms (FIG. 8). In this study, only positive or neutral charged peptide were allowed to pass through RNA nanopore, whereas negative charged DNA or peptide were not allowed to pass through RNA nanopore, which may be due to the electrostatic repulsing force between RNA nanopore and negative charged DNA or peptide. To study the folding of protein and even sequencing protein, several attempts have been made such as adjusting pH and using the AAA+ unfoldase ClpX to slow down peptide translocation. So far, short peptide detection still has not been reported. In this study, short amino acids as short as only two have been successfully detected in our platform, which has potential for applying protein sequencing in the future.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A ribonucleic acid nanopore, comprising a plurality of RNA oligonucleotides assembled into nanotube having a hollow channel 1 to 10 nm in diameter.

2. The nanopore of claim 1, wherein at least one of the RNA oligonucleotides comprises a membrane-anchoring moiety.

3. The nanopore of claim 2, wherein the membrane-anchoring moiety comprises a cholesterol moiety.

4. The nanopore of claim 1, wherein the nanopore is incorporated in and defines a channel through a lipid membrane.

5. The nanopore of claim 1, wherein the nanopore comprises four or more double helices, wherein each helix is formed by complementary base paring of two different RNA oligonucleotides.

6. The nanopore of claim 1, wherein each RNA oligonucleotide comprises at least two helix-forming regions that are complementary to helix-forming regions on distinct RNA oligonucleotides.

7. The nanopore of claim 1, wherein the RNA oligonucleotides comprise at least the nucleic acid sequences SEQ ID NO:1, SEQ ID NO:2, and SEQ ID NO:3.

8. The nanopore of claim 1, wherein the RNA oligonucleotides comprise the nucleic acid sequences SEQ ID NO:1, SEQ ID NO:2, SEQ ID NO:3, SEQ ID NO:4, SEQ ID NO:5, and SEQ ID NO:6.

9. The nanopore of claim 1, wherein the RNA oligonucleotides comprise the nucleic acid sequence SEQ ID NO:1, SEQ ID NO:2, SEQ ID NO:3, and SEQ ID NO:7.

10. The nanopore of claim 1, wherein the RNA oligonucleotides comprise the nucleic acid sequence SEQ ID NO:1, SEQ ID NO:2, SEQ ID NO:3, SEQ ID NO:4, and SEQ ID NO:8.

11. The nanopore of claim 1, wherein the RNA oligonucleotides comprise the nucleic acid sequence SEQ ID NO:1, SEQ ID NO:2, SEQ ID NO:3, SEQ ID NO:4, SEQ ID NO:5, SEQ ID NO:9, and SEQ ID NO:10.

12. The nanopore of claim 1, wherein the RNA oligonucleotides comprise the nucleic acid sequence SEQ ID NO:1, SEQ ID NO:2, SEQ ID NO:3, SEQ ID NO:4, SEQ ID NO:5, SEQ ID NO:9, SEQ ID NO:11, and SEQ ID NO:12.

13. The nanopore of claim 1, wherein the RNA oligonucleotides comprise the nucleic acid sequence SEQ ID NO:13, SEQ ID NO:14, SEQ ID NO:15, SEQ ID NO:16, SEQ ID NO:17, and SEQ ID NO:18.

14. The nanopore of claim 1, wherein the nanopore is about 5 to about 20 nm in length.

15. A method for sequencing a DNA, RNA, or protein sequence, comprising
(a) inserting an RNA nanotube into a lipid bilayer;
(b) inserting the lipid bilayer into an electrically resistive chip;
(c) applying an electric potential across the membrane to produce a current flowing only through the RNA nanotube;
(d) passing a single-stranded or double-stranded DNA, RNA, or protein strand through the RNA nanotube; and
(e) measuring a disruption in the current specific for each nucleotide or amino acid.

16. A method for detecting a molecule, comprising
(a) providing an RNA nanotube comprising a binding moiety specific for the molecule;
(b) inserting the RNA nanotube into a lipid bilayer;
(c) inserting the lipid bilayer into an electrically resistive chip;
(d) applying an electric potential across the membrane to produce a current flowing only through the RNA nanotube; and
(e) measuring a disruption in the current when the molecule is captured by the binding moiety of the RNA nanotube.
